(19) United States
(12) Patent Application Publication
(10) Pub. No.: US 2016/0215319 A1
(43) Pub. Date: Jul. 28, 2016

(54) IMPROVED PROCESS FOR PRODUCTION OF MONOCLONAL ANTIBODIES

(71) Applicant: CADILA HELTHCARE LIMITED, Ahmedabad, Gujarat (IN)

(72) Inventors: Sanjeev Kumar MENDIRATTA, Ahmedabad, Gujarat (IN); Sanjay BANDYOPADHYAY, Ahmedabad, Gujarat (IN); Sanjay PATEL, Ahmedabad, Gujarat (IN)

(73) Assignee: CADILA HELTHCARE LIMITED, Ahmedabad, Gujarat (IN)

(21) Appl. No.: 14/903,093

(22) PCT Filed: Jul. 7, 2014
(86) PCT No.: PCT/IN2014/000450

§ 371 Date: Jan. 6, 2016

(30) Foreign Application Priority Data
Jul. 6, 2013 (IN) 2285/MUM/2013

(51) Int. Cl. C12P 21/00; C07K 16/28; C07K 16/22; C07K 16/24; C07K 16/32

(52) U.S. Cl. CPC C12P 21/005; C07K 16/241; C07K 16/32; C07K 16/22; C07K 16/2887; C07K 2317/14

(57) ABSTRACT

The present invention provides for an improved process to obtain substantial amount of monoclonal antibodies with desired profile of charged variants. The process involves initially culturing e mammalian cells at a suitable temperature and subsequently reducing the temperature and optionally by simultaneous addition of suitable amino acid(s) during production of the desired molecule. The present invention provides also provides for an antibody having desired profile of glycans prepared with said with improved process.

### FIELD OF THE INVENTION

The present invention relates to an improved process to obtain substantial amount of monoclonal antibodies with desired profile of charged variants. In an embodiment, the process also provides an antibody with desired profile of glycans. The process involves initially culturing the mammalian cells at a suitable temperature and subsequently reducing the temperature and optionally by simultaneous addition of suitable amino acid(s) during production of the desired molecule.

### BACKGROUND OF THE INVENTION

Proteins are large and complex molecules. They are required to be in their native confirmation in order to remain biologically active. Further, at high concentration, protein molecules in solution are susceptible to undergo aggregation or degradation or certain modifications with time during storage. In one aspect, the present invention provides an improved method to obtain increased amount of desired quality product, preferably, monoclonal antibody. Monoclonal antibodies (mAbs) have gained significant attention as therapeutic agents due to their high degree of specificity in binding to the target antigens, ability to initiate immune response to the target antigen and long serum persistence. There are a number of monoclonal antibodies that are directed against tumor specific antigens. Some unique characteristic features of each of the immunoglobulins e.g. charge property and glycan structures are found to be important and specific for the mode of actions. Monoclonal antibodies like many other proteins have charge heterogeneity which optimize electrostatic interactions and regulates their structure, stability, chemical and biological properties. During production, various forms of micro heterogeneity occur due to degradation, modification or various enzymatic processes. Degradation of protein takes place due to chemical instability or physical instability. Chemical instability majorly can be result of deamidation, racemization, hydrolysis, oxidation, beta elimination or disulfide exchange. Chemical instability results in the formation of various charge variants and thus modifying the properties of the biomolecules. Chemical modification such as deamidation and sialylation, respectively, result in the increase in the net negative charge on mAbs and causes a decrease in pI values. Other mechanisms of generation of acidic variants are known in the prior art. Deamidated isoforms are susceptible to degrade with the loss of activity and therefore, it impacts significantly activity as well as stability of monoclonal antibody proteins.

Similarly, N-glycosylation in the Fc region modulates antibody effector functions of immunoglobulins and other Fc containing molecules. Fc glycans may contain several different types of terminal sugars that affect functions of antibodies. Effect of terminal galactosylation is known to the skilled person. Galactosylation pattern of different immunoglobulins shows product-specific variability in such immunoglobulins. It is important to note that variation in the terminal galactosylation affects the antibody binding to antigen and does influence greatly the CDC activity of the molecule. On the other hand, varying degree of galactosylation is known to have less influence on ADCC activity, whereas afucosylation is extremely important for ADCC activity.

There are several production processes of monoclonal antibodies known in the art. Such processes include maintenance of osmolality, addition of salt together with reduction in temperature, etc.

U.S. Pat. No. 5,705,364 discloses cell culture processes for controlling the amount of sialic acid present on an oligosaccharide side chain of a glycoprotein by adding an alkanoic acid or a salt thereof to the culture at a concentration of about 0.1 mM to about 20 mM maintaining the osmolality of the culture at about 250 to about 600 mOsm and maintaining the temperature of the culture at a temperature about between 30° C. and 35° C.

U.S. Pat. No. 5,976,833 provide a method for improving productivity in the production of useful substances by animal cells. It discloses a method for animal cell culture to produce a desired substance, comprising the steps of (1) culturing animal cells at a temperature at which the animal cells can grow; and (2) culturing the animal cells at a lower temperature.

WO 2014035475 discloses a method for controlling the oligosaccharide distribution of a recombinantly-expressed protein comprising supplementing a cell culture media used in the recombinant expression of said protein with a yeast hydrolysate supplement and a plant hydrolysate supplement. It also discloses method for controlling the oligosaccharide distribution of an antibody by modulating asparagine amino acid concentration of the cell culture media; whereas the present invention does not involve supplementation of such hydrolysate in the culture media.

Although there is an availability of different processes for the production of monoclonal antibodies, still there is a need to establish a cell culture process for monoclonal antibody production, which consistently generates the desired level of charged variants and glycans profile without any significant batch-to-batch variation. Such process will also help in obtaining the monoclonal antibody proteins with desired charge and/or glycan profile. The present invention provides such an improved process of the monoclonal antibody production using modified cell culture method. The process according to the present invention does not include either addition of salt or maintenance of suitable osmolality. The present invention provides novel method for the production of monoclonal antibodies with desired profile of glycans and charged variants.

### SUMMARY OF INVENTION

The present invention provides an improved process to obtain substantial amount of monoclonal antibodies with desired profile of glycans and charged variants using modified cell culture method.

In one aspect, cell culture method is characterized by maintaining the cell culture production condition at various temperatures either at once or in a step-wise manner during the cell culture process.

In another aspect, the present invention provides an improved process for the production of monoclonal antibody by carrying out the production process at an initial higher temperature in the growth phase and subsequently reducing the temperature of the culture system to a second lower temperature either during the mid-log to late-log phase or the stationary phase.

In another aspect, the present invention provides an improved process for the production of monoclonal antibody by feeding suitable amino acids to the culture system during the mid-log to late-log phase or the stationary phase.

In further aspect, according to the present invention the amino acid(s) are added to the cell culture medium at certain concentrations and at specific time-intervals during cell culture process.

In a preferred embodiment the amino acids are selected from glutamine and asparagine or combinations thereof.

In a preferred embodiment, the present invention provides an improved upstream process to obtain substantial amount of monoclonal antibodies with desired profile of glycans and charged variants by carrying out the process at an initial higher temperature in the growth phase, and subsequently, reducing the temperature of the culture system to a second lower than the initial temperature either during the mid-log to late-log phase or at the stationary phase with simultaneous feeding of amino acid(s) to the culture media.

In further aspect, the amino acid according to the present invention is selected from amide group containing and basic amino acids e.g. glutamine, asparagine, histidine, lysine, arginine and a combination thereof.

In a preferred embodiment, the monoclonal antibodies are selected from anti-HER antibody, anti-TNF antibody, anti-VEGF antibody and anti-CD20 antibody.

In a more preferred embodiment, the monoclonal antibodies are selected from trastuzumab, pertuzumab, infliximab, adalimumab, bevacizumab, ranibizumab and rituximab.

### BRIEF DESCRIPTION OF DRAWINGS

FIG. 1: Illustrates the charged variants profile of the purified Adalimumab protein by HP-IEC.

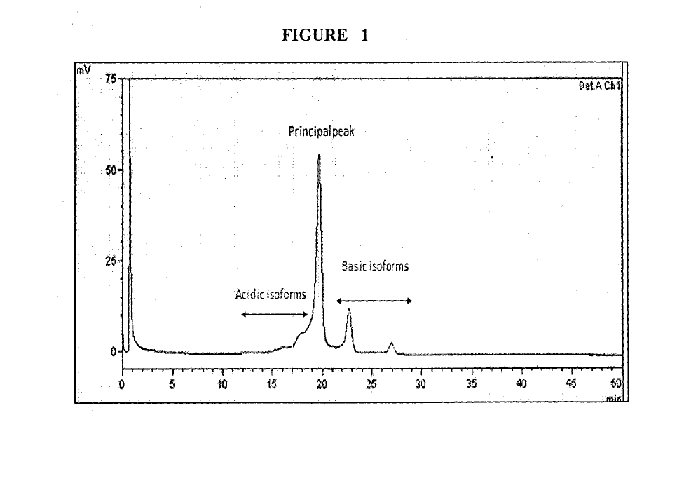

FIG. 2: Illustrates the glycans profile of the purified Adalimumab protein by CE-LIF.

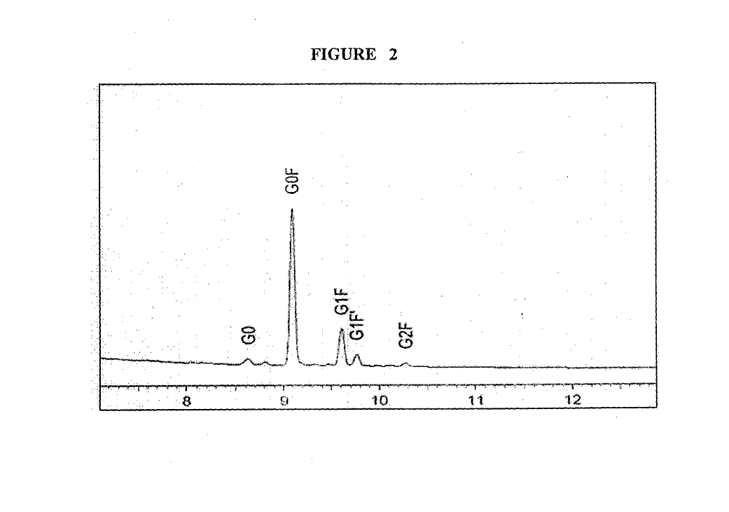

### DETAILED DESCRIPTION OF THE PRESENT INVENTION

In one embodiment, the present invention provides process for the to production of monoclonal antibody with desired profile of charged variants while maintaining the a desired glycan profile of the protein by carrying out the production process at an initial higher temperature in the growth phase, and subsequently, decreasing the temperature of the culture system to a lower temperature at once or in a step-wise manner, either during the mid-log to late-log phase or at the stationary phase.

In a further embodiment, cell culture method is characterized by maintaining the cell culture production condition at various temperatures either at once or in a step-wise manner during the cell culture process.

In another embodiment, the present invention provides a process for the production of substantial amount of monoclonal antibody with desired profile of glycans preferably by feeding suitable amino acids such as amide group containing amino acid(s) and/or basic amino acid(s) to the culture system. Such amino acid(s) can be fed at any stage during the mid-log phase to the stationary phase.

In further embodiment, according to the present invention the amino acid(s) are added to the cell culture medium at certain concentrations and at specific time-intervals during cell culture process.

In furthermore embodiment, according to the present invention addition of amino acid(s) is performed at least at two different intervals during the cell culture process.

In preferred embodiment, according to the present invention the addition of amino acid(s) to the cell culture medium is performed at concentration less than 20 mM, preferably less than 10 mM each time.

In a preferred embodiment, the present invention provides substantial amount of monoclonal antibodies with desired profile of glycans and charged variants by carrying out the production process at an initial higher temperature in the growth phase, and subsequently, decreasing the temperature of the culture system to a second lower temperature either during the mid-log to late-log phase or at the stationary phase and feeding of amino acid(s) to the culture system.

In further aspect, the amino acid according to the present invention is selected from amide group containing and basic amino acids e.g. glutamine, asparagine, histidine, lysine, arginine and a combination thereof.

Generally, the initial higher temperature of the culture system is maintained at 37° C. The temperature of the culture system according to the present invention can be decreased up to 30° C. either at once or step-wise at specific time interval, at any stage during the mid-log phase to stationary phase. The process according to the present invention provides substantial amount of monoclonal antibodies with desired profile of glycans and charged variants. Furthermore, the process maintains the desired glycans profile of the monoclonal antibody.

In one of the embodiments, the present invention provides desired glycans profile of the protein, preferably, monoclonal antibody by feeding suitable amino acids such as glutamine and/or asparagine to the culture system at any stage during the mid-log phase to stationary phase. The amount of amino acid(s) added is in the range of 1 to 4 mM, preferably 2 to 3 mM. Feeding of glutamine and/or asparagine according to the present invention to the cell culture media during production was found to augment the formation of the desired glycan(s) moiety(ies) in product-specific manner in monoclonal antibody protein structure.

In a preferred embodiment, the present invention provides production of substantial amount of monoclonal antibody with desired profile of glycans and charged variants by carrying out the process at an initial higher temperature in the growth phase, and subsequently, decreasing the temperature of the culture system to a second lower temperature either during the mid-log to late-log phase or at the stationary phase and by addition of suitable amino acid(s) (glutamine and/or asparagine) during production of the desired protein.

In one of the embodiments, the present invention provides a process for the production of antibody with desired profile of glycans and charged variants where glucose concentration is maintained in the range of 0.5 g/L to 8 g/L, preferably 2 g/L to 4 g/L, more preferably about 2.5 g/L.

In another embodiment, the present invention provides a process for the production of antibody with desired profile of glycans and charged variants where pH is maintained during production in the range of pH 6 to pH 7.5 by using suitable buffer selected from sodium bicarbonate, sodium carbonate and HEPES buffer.

In a further embodiment, the present invention provides a process for the production of antibody with desired profile of glycans and charged variants where cell productivity is maintained not less than 0.5 g/L, preferably 1-4 g/L.

In furthermore embodiment, the present invention provides a process for the production of antibody with desired profile of glycans and charged variants where cell viability is maintained not less than 30%, preferably about 80%, more preferably greater than 95%.

In more preferred embodiment, the monoclonal antibodies are selected from trastuzumab, pertuzumab, infliximab, adalimumab, bevacizumab, ranibizumab and rituximab.

### Definitions:

Glycan—The term glycan refers to a polysaccharide or oligosaccharide. Glycans can be homo- or heteropolymers of monosaccharide residues, and can be linear or branched. Glycan may also be used to refer to the carbohydrate portion of a glycoconjugate, such as a glycoprotein, glycolipid, or a proteoglycan.
Desired Glycan profile—It can be defined as distribution pattern of the various glycan molecules attached to the protein which are essential for its biological activity.
Mid-log phase—It is defined as the growth phase of cells in a culture medium during which cell population increases exponentially. This phase is represented by a part of the growth curve, which appears as a straight line segment when the logarithmic values of the cell population are plotted against time, called as logarithmic phase, and the mid-point of which is called as the mid-log phase.
Late-log phase—It is defined as the growth phase of cells at the late-log phase prior to the transition to the stationary phase. This phase is represented by a part of the growth curve, which appears as a straight line segment when the logarithmic values of the cell population are plotted against time, called as logarithmic phase, and the end phase of which is called as the late-log phase.
Stationary phase—The plateau of the growth curve after the log-phase growth of cells in culture medium, at which time the cell population remains constant is called as the stationary phase. New cells are produced at the same rate as older cells die.
Charge species variants—It is specific property of proteins which optimize electrostatic interactions and regulates their structure, stability, chemical and biological properties. It varies from protein to protein due to specific distribution of charged amino acids onto the protein molecule.
   Analytical Methods used in the Present Invention:

High Pressure Ion Exchange Chromatography (HP-IEC): Separation of different charged variants of the purified monoclonal antibody e.g. Adalimumab is performed by using an analytical HP-weak cation exchange chromatography. The column is equilibrated in sodium phosphate buffer of pH 6.9 (mobile phase A). Elution of the charged species variants of the said protein is carried out with increasing salt concentration (sodium chloride) in mobile phase A at 0.5 mL/min.

Capillary Electrophoresis-Laser Induced Fluorescence (CE-LIF): Glycan analysis (glycosylation variants) of the purified monoclonal antibody preparation, e.g. Adalimumab is conducted by CE-LIF method after isolating the carbohydrate moieties from the said protein by PNGase treatment. Following the enzymatic treatment, the carbohydrate (glycans) moieties are labeled by APTS (8-aminopyrene 1,2,6-trisulfonate) and the derivatized glycans are then separated by the capillary system (N-CHO coated; 50 cm×50 μm) on the basis of the hydrodynamic size. Glycans are identified against labeled glucose ladder standard detected by a LIF detector with an excitation wavelength of 488 nm and an emission wavelength of 520 nm.

The preferred manner of production process of the monoclonal antibody according to the present invention is illustrated below by the following examples which should not be interpreted as limiting the scope of the invention in any way:

### EXAMPLE 1

Mammalian cells expressing anti-TNFα antibody adalimumab were generated by standard molecular biology techniques. Clones were subjected to limiting dilution to obtain a single cell derived homogenous population. The cells were cryopreserved in the form of cell banks and used for further development. Cells were revived and propagated with a series of inoculum development steps and inoculated in the bioreactor containing suitable growth media. Cell culture was performed in a controlled environment by maintaining pH 7.2±0.4 using CO₂ gas and/or sodium bicarbonate, as and when required. The dissolved oxygen concentration was maintained at 40±20% saturation with sparging of air and/or oxygen gas and by controlling agitation speed in the bioreactor. Temperature was controlled at 37° C. Growth media contains following components:

| Components | Concentration |
|---|---|
| CHO growth powder media | 19.8 g/L |
| Sodium bicarbonate | 2.2 g/L |
| Pluronic F-68 | 1.2 g/L |

Cells were grown under the above mentioned conditions for two days. From day 3, feeding was initiated and continued till the end of batch. Following media components were fed to the cell culture medium as common feed—

| Components | Concentration per liter | |
|---|---|---|
| CHO basal powder media | 138.9 | g |
| Insulin | 50 | mg |
| Lipid supplement | 1x concentration | |
| Ferric citrate | 1x concentration | |
| Polyethylene glycol | 220 | mg |
| Sodium bicarbonate | 10.8 | g |

The batch was harvested between 13 and 18 days of culture. After cell clarification, the supernatant containing adalimumab was reconditioned to match substantially to the next purification column equilibration conditions. The desired protein was purified up to satisfactory level and submitted to HP-IEC and CE-LIF analysis for charged species variants and glycans profile, respectively, as shown in Table 1 and Table 2. Process exemplified here can be used for any desired antibody.

### EXAMPLE 2

Effect of Decreasing Temperature to 35° C. from 37° C. for Adalimumab

The experiment was carried out in a 30 L bioreactor. The growth conditions were identical to example-1 including the common feed media and other process parameters except that of the temperature conditions of the culture system. The temperature of the culture system was decreased from 37° C. to 35° C. at the late log phase. Adalimumab was purified up to satisfactory level and submitted to HP-fEC and CE-LIF analysis for charged species variants and glycans profile, respectively, as shown in Table 1 and Table 2.

### EXAMPLE 3

### Effect of Feeding of Glutamine for Adalimumab

The experiment was carried out in a 30 L bioreactor. The growth conditions were identical to example-1 including the common feed media and other process parameters except that of the feeding of glutamine amino acids to the culture system. The feeding of 2 mM glutamine was started at the mid-log-phase of cell growth and was continued till the end of production at specific intervals.

Adalimumab was purified up to satisfactory level and submitted to HP-IEC and CE-LIF analysis for charged species variants and glycans profile, respectively, as shown in Table 1 and Table 2.

### EXAMPLE 4

### Effects of Decreasing Temperature and Glutamine-Feed Adalimuinab

The experiment was carried out in a 30 L bioreactor. The growth conditions were identical to example-1 including the common feed media and other process parameters except that of the temperature condition and feeding of glutamine to the culture system. Temperature of the culture system was decreased from 37° C. to 35° C. at the mid log phase, after which time temperature of the culture system was further decreased to 33° C. during the transition from log-phase to stationary phase. Feeding of 3 mM glutamine was started at the mid-log phase and was continued at specific intervals till the end of production of the desired monoclonal antibody.

Adalimumab was purified up to satisfactory level and submitted to HP-IEC and CE-LIF analysis for charged species variants and glycans profile, respectively, as shown in Table 1 and Table 2.

### EXAMPLE 5

### Effect of Cultivating at Temperature 37° C. Without Feeding of Glutamine for Trastuzumab:

The experiment was carried out in a bioreactor. The growth conditions were identical to example-1 including the common feed media and other process parameters except that of the temperature condition and feeding of glutamine to the culture system. Temperature of the culture system was maintained at 37° C. throughout the batch duration. No further feeding of glutamine was done other than that in the initial batch media.

Trastuzumab was purified up to satisfactory level and submitted to HP-IEC and CE-LIF analysis for charged species variants and glycans profile, respectively, as shown in Table 1 and Table 2.

### EXAMPLE 6

Effect of Decreasing Temperature to 33° C. from 37° C. for Trastuzumab:

The experiment was carried out in a bioreactor. The growth conditions were identical to example-1 including the common feed media and other process parameters except that of the temperature condition and feeding of glutamine to the culture system. Temperature of the culture system was decreased from 37° C. to 33° C. during the transition from log-phase to stationary phase. Feeding of 2 mM glutamine was started at the mid-log phase and was continued at specific intervals till the end of production of the desired monoclonal antibody.

Trastuzumab was purified up to satisfactory level and submitted to HP-IEC and CE-LIF analysis for charged species variants and glycans profile, respectively, as shown in Table 1 and Table 2.

### EXAMPLE 7

Effect of Decreasing Temperature from 37° C. to 35° C. Without Feeding of Glutamine for Bevacizumab:

The experiment was carried out in a 30 L bioreactor (culti-flask). The growth conditions were identical to example-1 including the common feed media and other process parameters except that of the temperature condition and feeding of glutamine to the culture system. Temperature of the culture system was decreased from 37° C. to 35° C. during the transition from log-phase to stationary phase. No further feeding of glutamine was done other than that in the initial batch media.

Bevacizumab was purified up to satisfactory level and submitted to HP-IEC and CE-LIF analysis for charged species variants and glycans profile, respectively, as shown in Table 1 and Table 2.

### EXAMPLE 8

### Effect of Cultivating at 37° C. Throughout the Batch and Feeding of Glutamine for to Bevacizumab:

The experiment was carried out in a bioreactor. The growth conditions were identical to example-1 including the common feed media and other process parameters except that of the temperature condition and feeding of glutamine to the culture system. Temperature of the culture system was maintained at 37° C. during the entire batch. Feeding of 4 mM glutamine was started at the mid-log phase and was continued at specific intervals till the end of production of the desired monoclonal antibody.

Bevacizumab was purified up to satisfactory level and submitted to HP-IEC and CE-LIF analysis for charged species variants and glycans profile, respectively, as shown in Table 1 and Table 2.

### EXAMPLE 9

### Effect of Cultivating at 37° C. Throughout the Batch No Feeding of Glutamine for Rituximab:

The experiment was carried out in a bioreactor. The growth conditions were identical to example-1 including the common feed media and other process parameters except that of the temperature condition and feeding of glutamine to the culture system. Temperature of the culture system was maintained at 37° C. during the entire batch. No further feeding of glutamine was done other than that in the initial batch media.

Rituximab was purified up to satisfactory level and submitted to HP-IEC and CE-LIF analysis for charged species variants and glycans profile, respectively, as shown in Table 1 and Table 2.

### EXAMPLE 10

Effect of Cultivating at 37° C. Throughout the Batch with Feeding of Glutamine for Rituximab:

The experiment was carried out in a bioreactor. The growth conditions were identical to example-1 including the common feed media and other process parameters except that of the temperature condition and feeding of glutamine to the culture system. Temperature of the culture system was maintained at 37° C. during the entire batch. Feeding of 4 mM glutamine was started at the mid-log phase and was continued at specific intervals till the end of production of the desired monoclonal antibody.

Rituximab was purified up to satisfactory level and submitted to HP-IEC and CE-LIF analysis for charged species variants and glycans profile, respectively, as shown in Table 1 and Table 2.

### EXAMPLE 11

Effect of Cultivating at 37° C. Throughout the Batch with Feeding of Glutamine for Trastuzumab:

The experiment was carried out in a 30 L bioreactor (200 L bioreactor). The growth conditions were identical to example-1 including the common feed media and other process parameters except that of the temperature condition and feeding of glutamine to the culture system. Temperature of the culture system was maintained at 37° C. during the entire batch. Feeding of 2 mM glutamine was started at the mid-log phase and was continued at specific intervals till the end of production of the desired monoclonal antibody.

Trastuzumab was purified up to satisfactory level and submitted to HP-IEC and CE-LIF analysis for charged species variants and glycans profile, respectively, as shown in Table 1 and Table 2.

### EXAMPLE 12

Effect of Decreasing Temperature from 37° C. to 35° C. Without Feeding of Glutamine for Trastuzumab:

The experiment was carried out in a 30 L bioreactor (culti-flask). The growth conditions were identical to example-1 including the common feed media and other process parameters except that of the temperature condition and feeding of glutamine to the culture system. Temperature of the culture system was decreased from 37° C. to 35° C. during the transition from log-phase to stationary phase. No further feeding of glutamine was done other than that in the initial batch media.

Trastuzumab was purified up to satisfactory level and submitted to HP-IEC and CE-LIF analysis for charged species variants and glycans profile, respectively, as shown in Table 1 and Table 2.

### Results

TABLE 1
Effect of temperature on charged species variants
of various monoclonal antibodies

| | | | Charged species variants | | |
|---|---|---|---|---|---|
| | | | | Acidic | Basic |
| Example | | Glutamine | Principal | isoforms | isoforms |
| No. | Temperature | feeding | variant (%) | (%) | (%) |
| Example-1 | 37° C. | No | 69.92 | 15.49 | 14.59 |
| Example-2 | 37° C. to 35° C. | No | 75.37 | 5.22 | 19.42 |
| Example-3 | 37° C. | Yes | 74.67 | 10.54 | 14.80 |
| Example-4 | 37° C. to 35° C. to | Yes | 75.31 | 7.48 | 17.16 |
| | 33° C. | | | | |
| Example-5 | 37° C. | No | 75.96 | 14.33 | 9.71 |
| Example-6 | 37° C. to 33° C. | Yes | 77.46 | 11.41 | 11.11 |
| Example-7 | 37° C. to 35° C. | No | 68.53 | 24.76 | 6.70 |
| Example-8 | 37° C. | Yes | 66.34 | 14.55 | 14.35 |
| Example-9 | 37° C. | No | 69.98 | 10.53 | 19.48 |
| Example-10 | 37° C. | Yes | 62.43 | 5.89 | 31.68 |
| Example-11 | 37° C. | Yes | 74.64 | 17.68 | 7.67 |
| Example-12 | 37° C. to 35° C. | No | 83.79 | 11.29 | 4.93 |

TABLE 2
Effect of glutamine feeding on glycans profile
of various monoclonal antibodies

| | | | Glycan profile (%) | |
|---|---|---|---|---|
| | | | | Galactose |
| | | | Agalactose | moiety |
| | | | moiety | (G1F, |
| | | Glutamine | (G0 and | G1′F and |
| Example No. | Temperature | feeding | G0F) | G2F) |
| Example-1 | 37° C. | No | 69.14 | 30.86 |
| Example-2 | 37° C. to 35° C. | No | 67.36 | 32.64 |
| Example-3 | 37° C. | Yes | 85.46 | 14.54 |
| Example-4 | 37° C. to 35° C. | Yes | 75.49 | 24.51 |
| | to 33° C. | | | |
| Example-5 | 37° C. | No | 69.26 | 30.74 |
| Example-6 | 37° C. to 33° C. | Yes | 73.76 | 26.24 |
| Example-7 | 37° C. to 35° C. | No | 66.72 | 23.28 |
| Example-8 | 37° C. | Yes | 81.40 | 18.42 |
| Example-9 | 37° C. | No | 64.16 | 35.84 |
| Example-10 | 37° C. | Yes | 79.00 | 21.00 |
| Example-11 | 37° C. | Yes | 65.934 | 34.065 |
| Example-12 | 37° C. to 35° C. | No | 51.40 | 34.103 |

The obtained product is subsequently purified and suitably formulated by techniques known in the art.

1-13. (canceled)

14. A process for producing an antibody with glycans and/or charge species variants profiles essential for its biological activity using modified cell culture method wherein said method comprises:
a) maintaining cell culture conditions at temperature conditions in between 30° C. to 37° C. during growth phase to stationary phase;
b) simultaneously or sequentially adding amino acid(s) to the culture medium during growth phase to stationary phase
wherein the antibody is selected from anti-HER antibody, anti-TNF antibody, anti-VEGF antibody and anti-CD20 antibody,

15. The process as claimed in claim 14, wherein the cell culture method is characterized by maintaining the cell culture production condition at temperatures between 30° C. to 37° C. either at a fixed temperature or reduction in temperature in a step-wise manner during the cell culture process.

16. The process as claimed in claim 15, wherein cell growth is at a first temperature condition up to the mid-log phase followed by maintaining the cell culture condition at a second and, optionally, a third temperature conditions until the end of the process.

17. The process as claimed in claim 16, wherein the first temperature condition is maintained at a temperature higher than the second and, optionally, the third temperature conditions.

18. The process as claimed in claim 16, wherein the first temperature condition is about 37° C. and the second temperature is in, a range of 30° C. to 35° C.

19. The process as claimed in claim 16 wherein the third temperature condition is in a range of 30° C. to 35° C.

20. The process as claimed in claim 14, wherein addition of amino acid(s) is carried out at least at two different intervals during the cell culture process.

21. The process as claimed in claim 14, wherein the addition of amino acid(s) to the cell culture medium is performed at a concentration of less than 10 mM each time.

22. The process as claimed in claim 14, wherein the amino acid(s) is selected from amide group containing basic amino acids.

23. The process, as claimed in claim 14, wherein the amino acid is selected from glutamine, asparagine, histidine, lysine, and arginine.

24. The process as claimed in claim 14, wherein glucose concentration is maintained in the range of 0.5 g/L to 8 g/L.

25. The process as claimed in claim 14, wherein glucose concentration is maintained in the range of 2 g/L to 4 g/L.

26. The process as claimed in claim 14, wherein pH is maintained in the range of pH 6 to pH 7.5 by using suitable buffer selected from sodium bicarbonate, sodium carbonate and HEPES buffer.

27. The process as claimed in claim 14, wherein cell productivity is not less than 0.5 g/L.

28. The process as claimed in claim 14, wherein cell productivity is not less than 1-4 g/L.

29. The process as claimed in claim 14 wherein cell viability is maintained at not less than 30%.

30. The process as claimed in claim 14, wherein cell viability is maintained at 80%.

31. The process as claimed in claim 14, wherein cell viability is maintained more than 95%.

32. The process as claimed in claim 14, wherein an antibody is selected from trastuzumab, pertuzumab, adalimumab, bevacizumab, ranibizumab and rituximab.
